(19) United States
(12) Patent Application Publication
(10) Pub. No.: US 2014/0005553 A1
(43) Pub. Date: Jan. 2, 2014

(54) SYSTEMS AND METHODS FOR ANALYSIS AND TREATMENT OF A BODY LUMEN

(71) Applicants: S. Eric Ryan, Hopkinton, MA (US); Jing Tang, Arlington, MA (US); Richard Gambale, Tyngsboro, MA (US)

(72) Inventors: S. Eric Ryan, Hopkinton, MA (US); Jing Tang, Arlington, MA (US); Richard Gambale, Tyngsboro, MA (US)

(73) Assignee: CORNOVA, INC., West Medford, MA (US)

(21) Appl. No.: 13/824,631

(22) PCT Filed: Sep. 21, 2011
(86) PCT No.: PCT/US11/52609

§ 371 Date: Aug. 9, 2013

Related U.S. Application Data
(60) Provisional application No. 61/385,013, filed on Sep. 21, 2010.
(60) Provisional application No. 61/444,502, filed on Feb. 18, 2011.

(51) Int. Cl. A61B 5/00; A61B 5/02; A61B 5/107

(52) U.S. Cl. CPC A61B 5/0086; A61B 5/0075; A61B 5/1076; A61B 5/02007; A61B 5/4836
USPC 600/473

(57) ABSTRACT

A system is provided for probing a body lumen that includes a flexible conduit that is elongated along a longitudinal axis, the flexible conduit having a proximal end and a distal end, at least one delivery waveguide and at least one collection waveguide extending along the flexible conduit, a transmission output of the at least one delivery waveguide and a transmission input of the at least one collection waveguide located along a distal portion of the conduit. A spectrometer is connected to the at least one delivery waveguide and the at least one collection waveguide, the spectrometer configured to perform spectroscopy. A controller system is configured to calculate a distance between the flexible conduit and the wall of the body lumen based on a spectroscopic measurement of the at least one primary radiation signal that traveled between the flexible conduit and body lumen.

### RELATED APPLICATIONS

This application claims the benefit of U.S. Provisional Patent Application No. 61/385,013 filed Sep. 21, 2010 and U.S. Provisional Patent Application No. 61/444,502 filed Feb. 18, 2011, the entire contents of each of which is herein incorporated by reference. This application is related to PCT Patent Application No. PCT/US2010/035677 filed May 20, 2010, U.S. patent application Ser. No. 12/784,482, filed May 20, 2010, U.S. Patent Application No. 61/180,068, filed May 20, 2009, U.S. Patent Application No. 61/310,337, filed Mar. 4, 2010, the entire contents of each of which is herein incorporated by reference. This application is related to U.S. patent application Ser. No. 11/834,096, filed on Aug. 6, 2007, published as U.S. Patent Application Publication No. 2007/0270717 A1, U.S. patent application Ser. No. 11/537,258, filed on Sep. 29, 2006, U.S. Patent Application No. 61/019,626, filed Jan. 8, 2008, U.S. Patent Application No. 61/025,514, filed Feb. 1, 2008, U.S. Patent Application No. 61/082,721 filed Jul. 22, 2008, U.S. patent application Ser. No. 12/350,870, filed Jan. 8, 2009, U.S. patent application Ser. No. 12/561,756, filed Sep. 17, 2009, the contents of each of which is incorporated herein in their entirety by reference.

### BACKGROUND OF THE INVENTION

1. Field of the Invention

Embodiments of inventive concepts are directed to systems and methods for the analysis and treatment of a lumen. More particularly, the present inventive concepts relate to a catheter probe systems used to perform methods of analysis including measuring the size and shape of lumens and can be performed in conjunction with angioplasty procedures.

2. Description of the Related Art

With the continual expansion of minimally-invasive procedures in medicine, one procedure that has been highlighted in recent years has been percutaneous transluminal angioplasty, or “PTA”. The most prevalent use of this procedure is in the coronary arteries, which is more specifically called a percutaneous coronary transluminal angioplasty, or “PTCA”. These procedures utilize a flexible catheter with an inflation lumen to expand, under relatively high pressure, a balloon at the distal end of the catheter to expand a stenotic lesion.

The PTA and PTCA procedures are now commonly used in conjunction with expandable tubular structures known as stents, and an angioplasty balloon is often used to expand and permanently place the stent within the lumen. An angioplasty balloon utilized with a stent is referred to as a stent delivery system. Conventional stents have been shown to be more effective than angioplasty alone in maintaining patency in most types of lesions and also reducing other near-term endovascular events. A risk with a conventional stent, however, is the reduction in efficacy of the stent due to the growth of the tissues surrounding the stent which can again result in the stenosis of the lumen, often referred to as restenosis. In recent years, new stents that are coated with pharmaceutical agents, often in combination with a polymer, have been introduced and shown to significantly reduce the rate of restenosis. These coated stents are generally referred to as drug-eluting stents, though some coated stents have a passive coating instead of an active pharmaceutical agent.

Studies have shown that having information about the morphology (including the size and shape) of a targeted lumen, both before and after stenting, can improve clinical outcomes, including avoidance of restenosis. See, e.g., “The IVUS Explosion, A Practical Guide: The Latest Data, The Latest Tips and Tricks,” Robert J. Russo, MD, PhD, Director, Intravascular Imaging and Cardiac MRI Programs, Scripps Clinic, La Jolla, Calif. 2009.

However, there are very few, if any, highly safe and commercially viable applications making use of combining diagnosis and treatment in a PTA or PTCA procedure. Some techniques include deployment of an additional catheter in order to both adequately examine a lumen and complete the desired treatment and/or ensure that an underexpanded stent is not blocking blood flow through a vessel. Additional procedures can result in increased risks and added expense. Accurate information about the apposition and expansion of the balloon and/or stent against the vessel walls while performing angioplasty procedures could therefore be highly useful for mitigating these risks.

Typical technologies used for monitoring angioplasty and stenting procedures include angiography by fluoroscopy, which supplies an X-ray image of the blood flow within a lumen. However, this technology has a very limited resolution of about 300 micrometers. As a result, many angioplasty and stenting procedures overexpand the lumen, which can result in unnecessary trauma and damage to the lumen wall, complicating post-deployment recovery, and increasing the likelihood of re-closure of the lumen (restenosis). For these reasons, stent deployment may be avoided altogether and substituted with less risky but less effective procedures.

Angioscope technology has also been attempted for examining a lumen during angioplasty but due to constraints on the numbers and sizes of fibers that can be placed within small vessels, only limited information can be gained from direct visualization. Other technologies, such as intravascular ultrasound (e.g., IVUS) and Optical Coherence Tomography (OCT), can require additional expensive or risky procedures. These technologies often do not provide consistent or accurate measurements of lumen characteristics and must be interpreted individually by an attending physician or technician, and thus increases the possibility of error. Studies have confirmed that data from these technologies is often misinterpreted leading to reduced efficacy. Conventional balloon catheters are not generally used for purposes other than for performing traditional angiosplasty procedures including pre-dilation of the vasculature prior to stent delivery, stent delivery, and post-stent delivery dilation. The availability of assessing the aforementioned pathophysiologic or morphologic factors while performing angioplasty procedures would be highly useful to enhance the safety and effectiveness of such treatments.

In certain applications, catheter probes have been proposed with angioplasty balloons, and rely on making use of the expanded balloon to displace blood from and clear the region of analysis (see, e.g., U.S. patent application No. Freeman). However, requiring deployment of the angioplasty balloon with analysis can increase risks (e.g., damage to the lumen wall), particularly where an angioplasty prior to analysis may not be indicated or necessary. Furthermore, deploying such a system with a crimped stent in place over the balloon can interfere with the optical view of the probe system.

There are currently needs for physicians to gain useful and more accurate information about the lumen wall, including obtaining accurate information about the size and shape of the pre-angioplasty and post-stented lumens in connection with angioplasty procedures in a cost-effective, and efficient manner that presents a reasonable risk profile for the patient.

### SUMMARY OF THE INVENTION

The systems and methods described in the present specification provide physicians performing a lumen-expansion procedure with very useful information about the lumen wall, including lumen size, without any significant increase in their procedure time or cost, and with little to no additional risk to the patient. Included are a number of implementations of distal fiber-optic configurations to optimally facilitate analysis of the lumen wall and angioplasty balloon characteristics. These implementations also provide manufacturability and relatively low-cost production required for a disposable medical device.

In an aspect of inventive concepts, a system for analyzing a body lumen is provided that includes a catheter having a flexible conduit that is elongated along a longitudinal axis, the flexible conduit having a proximal end and a distal end, at least one delivery waveguide and at least one collection waveguide extending along the flexible conduit, a transmission output of the at least one delivery waveguide and a transmission input of the at least one collection waveguide located along a distal portion of the conduit, a spectrometer connected to the at least one delivery waveguide and the at least one collection waveguide, the spectrometer configured to perform diffuse reflectance spectroscopy through blood, wherein the spectrometer emits at least one primary radiation signal of a wavelength of between about 750 and 2500 nm that is directed through the transmission output to a wall of the body lumen, and wherein the transmission input collects radiation directed from the body lumen wall, and a controller system including computer-readable memory programmed to store the signal measured by the spectrometer and to enable the controller to calculate a distance between the catheter and the wall of the body lumen based on a signal measured by the spectrometer of the at least one primary radiation signal that traveled through blood between the catheter and body lumen, the controller further programmed to store the calculated distance in the computer-readable memory.

In an embodiment, the spectrometer is further configured to perform spectroscopy of at least one reference radiation signal, and wherein the controller system is further programmed to calculate and store in the computer-readable memory a ratio of detected signals between the detected signal of the at least one primary radiation signal and a detected signal of the at least one reference radiation signal measured through blood by the spectrometer in order to calculate the distance between the flexible conduit and the wall of the body lumen.

In an embodiment, the at least one reference radiation signal includes a wavelength having an absorption coefficient in water of less than about 8 cm⁻¹. In an embodiment, the at least one reference radiation signal includes a wavelength having an absorption coefficient in water of between about 0.3 and 0.7 cm⁻¹. In an embodiment, the at least one reference radiation signal includes a wavelength of between about 1020 and 1120 nm. In an embodiment, the at least one reference radiation signal includes a wavelength of about 1060 nm. In an embodiment, the at least one reference radiation signal includes a wavelength of about 1310 nm. In an embodiment, the at least one primary radiation signal includes a wavelength of about 1060 nm.

In an embodiment, the computer-readable memory is programmed with an algorithm for enabling the controller to calculate a ratio of detected signals between the detected signal of the at least one primary radiation signal and an detected signal of at least one reference radiation signal and comparing the ratio to previously calculated and stored ratios measured from one or more catheters correspondingly configured to said catheter including a flexible conduit.

In an embodiment, the at least one delivery waveguide and at least one collection waveguide are arranged to measure the at least one primary radiation signal across a plurality of regions distributed about the circumference of the conduit and between the flexible conduit and the wall of the body lumen. In an embodiment, the computer-readable memory is programmed to enable the controller to calculate a cross-sectional area of the lumen from the measurements across the plurality of regions.

In an embodiment, the at least one primary radiation signal includes a wavelength having an absorption coefficient in water of between about 0.05 and 0.3 cm⁻¹. In an embodiment, the at least one primary radiation signal includes a wavelength between about 900 and 1000 nm.

In an embodiment, the at least one primary radiation signal includes a wavelength having an absorption coefficient in water of between about 0.7 and 1 cm⁻¹. In an embodiment, the at least one primary radiation signal includes a wavelength between about 1120 and 1150 nm.

In an embodiment, the at least one primary radiation signal includes a wavelength having an absorption coefficient in water of between about 0.3 and 0.7 cm⁻¹. In an embodiment, the at least one primary radiation signal includes a wavelength of between about 1020 and 1120 nm.

In an embodiment, the computer-readable memory is programmed with an algorithm that represents a multivariate analysis of preliminary measurements taken from one or more catheters correspondingly configured as said catheter including a flexible conduit. In an embodiment, the multivariate analysis includes at least one of multiple regression analysis, logistic regression analysis, discriminant analysis, multivariate analysis of variance, factor analysis, cluster analysis, multidimensional scaling, correspondence analysis, conjoint analysis, canonical correlation, and structural equation modeling.

In an embodiment, the catheter further includes a removable calibration sheath surrounding the transmission output of the at least one delivery waveguide and the transmission input of the at least one collection waveguide, the calibration sheath arranged to return radiation to the transmission input of the at least one collection waveguide in response to receiving radiation from the transmission output of the at least one delivery waveguide. In an embodiment, the calibration sheath includes a tissue phantom so as to permit simulation of delivering radiation from the transmission output to the tissue phantom and receiving radiation from the tissue phantom through the transmission input. In an embodiment, the tissue phantom includes at least one of an artificial blood phantom and artificial blood vessel wall phantom.

In an embodiment, the calibration sheath is arranged to improve the accuracy of the calculation of a distance between the catheter and the wall of the body lumen by the calculation of calibration factors that are programmed to be calculated by the controller and stored in computer-readable memory after operating the spectrometer with the calibration sheath in place over the catheter.

In an embodiment, the system further includes an angioplasty balloon disposed about a distal portion of the conduit. In an embodiment, the transmission output of the at least one delivery waveguide and the transmission input of the at least one collection waveguide is located within the angioplasty balloon.

In an embodiment, the at least one delivery waveguide and collection waveguide includes a fiber optic that has an end that operates as a reflection surface for changing a direction of a path of radiation to or from a direction transverse to the axis of the fiber optic.

In an embodiment, the end of the fiber optic includes a tip with a a core and a recess formed in said core at a distal end of the optical fiber tip to direct radiation transversely from the longitudinal axis of the fiber optic, said recess having a vertex within said core and the core having a maximum depth of less than about 70 microns.

In an embodiment, a first optical element disposed about the flexible conduit, the optical element including an array of multiple facets that lie at an acute angle relative to the longitudinal axis of the flexible conduit for changing a direction of radiation transmitted to or from a longitudinal axis of the at least one delivery or collection waveguide so that the radiation is emitted or collected to or from a direction that is transverse to the longitudinal axis of the at least one delivery or collection waveguide. In an embodiment, at least one of the multiple facets includes a width along the circumference of the flexible conduit that is at least 1.5 times the height of the at least one facet along the longitudinal direction of the flexible conduit. In an embodiment, the at least one of the multiple facets includes the shape of a concave parabola so as to further concentrate the delivery or collection of a signal across a longitudinal span of the lumen wall.

In an embodiment, the system further includes a second optical element for aligning distal ends of the at least one delivery or collection waveguide with the reflective facets of the first optical element. In an embodiment, the second optical element segment includes at least one feature for aligning the distal ends of the at least one delivery or collection waveguide with the reflective facets. In an embodiment, the at least one feature includes a shape having a plurality of flat sides arranged about the circumference of the conduit so as to rotationally align with the reflective facets.

In an embodiment, the second optical element includes at least one of holes or grooves extending along the entire longitudinal extent of the second optical element through which at least one of the at least one delivery waveguide and collection waveguide passes through. In an embodiment, the second optical element further includes an array of multiple facets that lie at an acute angle relative to the longitudinal axis of the flexible conduit for changing a direction of radiation transmitted to or from a longitudinal axis of at least one delivery or collection waveguide so that the radiation is emitted or collected to or from a direction that is transverse to the longitudinal axis of the at least one delivery or collection waveguide. In an embodiment, the facets of the first optical element are separated from the facets of the second optical element by a predetermined longitudinal distance. In an embodiment, the predetermined longitudinal distance is about 2.5 mm.

In an embodiment, at least one of the first and second optical elements is configured for delivering signals to an adjacent lumen and at least one of the first and second optical elements is configured for collecting signals from the adjacent lumen.

In an embodiment, at least one of the waveguides terminates at one of the multiple facets.

In an embodiment, the computer-readable memory of the controller is further programmed to enable the controller to measure at least one of the characteristics of plaque within a lumen wall including at least one of collagen content, lipid content, calcium content, inflammation, or the relative positioning of pathophysiologic conditions within the plaque.

In an aspect of inventive concepts, a method for providing analysis of a body lumen is provided, the method including the steps of inserting into a body lumen a catheter including a flexible conduit that is elongated along a longitudinal axis, the flexible conduit having a proximal end and a distal end, at least one delivery waveguide and at least one collection waveguide extending along the flexible conduit, and a transmission output of the at least one delivery waveguide and a transmission input of the at least one collection waveguide located along a distal portion of the conduit, maneuvering the conduit into a designated region of the body lumen designated for treatment or analysis, executing spectroscopic analysis of the designated region of the body lumen using at least one primary radiation signal having a wavelength in a range of about 750 to 2500 nanometers by radiating the designated region of the body lumen with supplied radiation that is supplied at the transmission output of the at least one delivery waveguide, the supplied radiation incident on the designated region of the body lumen, and wherein radiation is returned from the body lumen to the transmission input of the at least one collection waveguide, calculating a distance between the catheter and the wall of the body lumen based on radiation measured from the spectroscopic analysis of the at least one primary radiation signal that traveled through blood between the catheter and body lumen, and storing the calculated distance in computer-readable memory.

In an embodiment, executing spectroscopic analysis further includes spectroscopic analysis of at least one reference radiation signal of a wavelength having an absorption coefficient of less than about 8 cm⁻¹ in water and wherein the calculating a distance includes calculating a ratio of a detected signal of the at least one primary radiation signal and a detected signal of the at least one reference radiation signal measured through blood between the flexible conduit and the wall of the body lumen. In an embodiment, the at least one reference radiation signal includes a wavelength having an absorption coefficient in water of less than about 8 cm⁻¹. In an embodiment, the at least one reference radiation signal includes a wavelength having an absorption coefficient in water of between about 0.3 and 0.7 cm⁻¹. In an embodiment, the at least one reference radiation signal includes a wavelength of between about 1020 and 1120 nm. In an embodiment, the at least one reference radiation signal includes a wavelength of about 1060 nm.

In an embodiment, the at least one reference radiation signal includes a wavelength of about 1310 nm. In an embodiment, the at least one primary radiation signal includes a wavelength of about 1060 nm.

In an embodiment, the spectroscopic analysis of the at least one primary radiation signal is measured across a plurality of regions distributed about the circumference of the conduit and between the flexible conduit and the wall of the body lumen. In an embodiment, the method further includes calculating a cross-sectional area of the lumen from the measurements across the plurality of regions.

In an embodiment, the at least one primary radiation signal includes a wavelength having an absorption coefficient in water of between about 0.05 and 0.3 cm⁻¹. In an embodiment, the at least one primary radiation signal includes a wavelength between about 900 and 1000 nm.

In an embodiment, the at least one primary radiation signal includes a wavelength having an absorption coefficient in water of between about 0.7 and 1 cm⁻¹. In an embodiment, the at least one primary radiation signal includes a wavelength between about 1120 and 1150 nm.

In an embodiment, the at least one primary radiation signal includes a wavelength having an absorption coefficient in water of between about 0.3 and 0.7 cm⁻¹. In an embodiment, the at least one primary radiation signal includes a wavelength of between about 1020 and 1120 nm.

In an embodiment, the computer-readable memory is programmed with an algorithm that represents a multivariate analysis of preliminary measurements taken from one or more catheters correspondingly configured as said catheter including a flexible conduit. In an embodiment, the multivariate analysis includes at least one of multiple regression analysis, logistic regression analysis, discriminant analysis, multivariate analysis of variance, factor analysis, cluster analysis, multidimensional scaling, correspondence analysis, conjoint analysis, canonical correlation, and structural equation modeling.

In an embodiment, the method includes a step, prior to maneuvering the conduit into a designated region of the body lumen, executing spectroscopic analysis of a removable calibration sheath through the transmission output of the at least one delivery waveguide and the transmission input of the at least one collection waveguide, calculating and storing in computer-readable memory calibration factors based upon the spectroscopic analysis of the removable calibration sheath, and wherein the calculating a distance between the catheter and the wall of the body lumen is adjusted by the calibration factors. In an embodiment, the calibration sheath includes a tissue phantom through which the spectroscopic analysis of the removable calibration sheath is performed. In an embodiment, the tissue phantom includes at least one of an artificial blood phantom and artificial blood vessel wall phantom.

In an embodiment, the catheter includes an angioplasty balloon disposed about a distal portion of the conduit.

In an embodiment, the transmission output of the at least one delivery waveguide and the transmission input of the at least one collection waveguide is located within the angioplasty balloon. In an embodiment, an angioplasty procedure is performed by the angioplasty balloon and one or more parameters of the angioplasty procedure is determined by the calculated distance between the catheter and the wall of the body lumen. In an embodiment, the level of expansion of the angioplasty balloon is determined from a cross-sectional area of the lumen determined by calculating a distance between the catheter and the wall of the body lumen across a plurality of regions about the circumference of the conduit.

In an embodiment, the at least one delivery waveguide and collection waveguide includes a fiber optic that has an end that reflects the path of radiation surface for changing a direction of a path of radiation to or from a direction transverse to the axis of the fiber optic.

In an embodiment, the end of the fiber optic includes a tip with a a core and a recess formed in said core at a distal end of the optical fiber tip to direct radiation transversely from the longitudinal axis of the fiber optic, said recess having a vertex within said core and the core having a maximum depth of less than about 70 microns.

In an embodiment, a first optical element is disposed about the flexible conduit, the optical element including an array of multiple facets that lie at an acute angle relative to the longitudinal axis of the flexible conduit for changing a direction of radiation transmitted to or from a longitudinal axis of the at least one delivery or collection waveguide so that the radiation is emitted or collected to or from a direction that is transverse to the longitudinal axis of the at least one delivery or collection waveguide. In an embodiment, at least one of the multiple facets includes a width along the circumference of the flexible conduit that is at least 1.5 times the height of the at least one facet along the longitudinal direction of the flexible conduit.

In an embodiment, at least one of the multiple facets includes the shape of a concave parabola so as to further concentrate the delivery or collection of a signal across a longitudinal span of the lumen wall.

In an embodiment, the catheter further includes a second optical element for aligning distal ends of the at least one delivery or collection waveguide with the reflective facets of the first optical element. In an embodiment, the second optical element segment includes at least one feature for aligning the distal ends of the at least one delivery or collection waveguide with the reflective facets. In an embodiment, at least one feature includes a shape having a plurality of flat sides arranged about the circumference of the conduit so as to rotationally align with the reflective facets. In an embodiment, the second optical element includes at least one of holes or grooves extending along the entire longitudinal extent of the second optical element through which at least one of the at least one delivery waveguide and collection waveguide passes through. In an embodiment, the second optical element further includes an array of multiple facets that lie at an acute angle relative to the longitudinal axis of the flexible conduit for changing a direction of radiation transmitted to or from a longitudinal axis of at least one delivery or collection waveguide so that the radiation is emitted or collected to or from a direction that is transverse to the longitudinal axis of the at least one delivery or collection waveguide. In an embodiment, the facets of the first optical element are separated from the facets of the second optical element by a predetermined longitudinal distance. In an embodiment, the predetermined longitudinal distance is about 2.5 mm.

In an embodiment, at least one of the first and second optical elements delivers signals to an adjacent lumen and at least one of the first and second optical elements collects signals from the adjacent lumen.

In an embodiment, at least one of the waveguides terminates at one of the multiple facets.

In an embodiment, the method further includes measuring at least one of the characteristics of plaque within a lumen wall including at least one of collagen content, lipid content, calcium content, inflammation, or the relative positioning of pathophysiologic conditions within the plaque.

In an embodiment, the controller is further configured to measure at least one or more pathophysiologic or morphologic factors of surrounding tissue within an endovascular region. In an embodiment, the pathophysiologic or morphologic factors include characterizing the presence, volume, and positioning of plaque within the endovascular region. In an embodiment, the pathophysiologic or morphologic factors further include characteristics of plaque including at least one of collagen content, lipid content, calcium content, inflammation, or the relative positioning of pathophysiologic conditions within the plaque.

In an embodiment, the controller and spectrometer are configured to measure the at least one or more pathophysiologic or morphologic factors of surrounding tissue within an endovascular region by analyzing at least one wavelength less between about 750 nm and 1100 nm, and comparing the analysis of said at least one wavelength with the calculated distance between the catheter and the wall of the body lumen.

In an embodiment, the at least one wavelength between about 750 nm and 1100 nm includes 1060 nm.

Other advantages and novel features, including optical methods and designs of illuminating and collecting an optical signal of a lumen wall are described within the detailed description of the various embodiments of the present specification.

### BRIEF DESCRIPTION OF THE DRAWINGS

The foregoing and other objects, features, and advantages of the invention will be apparent from the more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1A is an illustrative view of a catheter instrument for analyzing and medically treating a lumen, according to an embodiment of inventive concepts.

FIG. 1B is a block diagram illustrating an instrument deployed for analyzing and medically treating the lumen of a patient, according to an embodiment of inventive concepts.

FIG. 2A is an illustrative side perspective view of a catheter tip-probe section according to an embodiment of inventive concepts.

FIG. 2B is a cross-sectional view of the tip-probe section of FIG. 2A, taken along section lines I-I′ of FIG. 2A.

FIG. 3A is an illustrative perspective view of a tip-probe section with 6 fibers according to another embodiment of inventive concepts.

FIG. 3B is an illustrative side-perspective view of the probe section of FIG. 3A.

FIG. 3C is a cross-sectional view of the tip-probe section of FIG. 3A taken along section lines I-I′ of FIG. 3B.

FIG. 3D is an illustrative perspective view of an alignment and reflector segment according to an embodiment of inventive concepts.

FIG. 3E is a side perspective view of the alignment and reflector segment of FIG. 3D.

FIG. 3F is a cross-sectional view of the alignment and reflector of FIG. 3D, taken along section lines I-I′ of FIG. 3E.

FIG. 3G is an illustrative side-perspective view of a tip-probe section of a catheter according to an embodiment of inventive concepts.

FIG. 3H is a cross-sectional view of a fiber tip of the catheter of FIG. 3G.

FIG. 3G is a side perspective view of a fiber and curve-shaped reflector according to an embodiment of inventive concepts.

FIG. 3H is another side-perspective view of a reflector incorporated into a multi-faceted reflective piece according to an embodiment of inventive concepts.

FIG. 3I is a side perspective view of a lumen illuminated by the fiber and reflector of FIGS. 3G and 3H.

FIG. 4A is a logarithmic chart of measured absorption coefficients in water relative to selected near-infrared wavelengths of light.

FIG. 4B is a chart of intensity measurement ratios measured through bovine blood at varying distances between a bovine blood vessel wall and catheter wall using wavelengths of 1310 nm and 1060 nm.

FIG. 4C is a chart of intensity measurement ratios measured through bovine blood at varying distances between a bovine blood vessel wall and catheter wall using wavelengths of 980 nm and 1060 nm.

FIG. 4D is an illustrative chart representing calculations made from exemplary absorbance signals according to embodiments of the invention.

FIG. 5A is an illustrative perspective view of a catheter probe deployed within and analyzing a blood vessel according to an embodiment of inventive concepts.

FIG. 5B is an illustrative perspective view of the catheter probe of FIG. 5A positioned for an angioplasty procedure according to an embodiment of inventive concepts.

FIG. 5C is an illustrative perspective view of the catheter probe of FIG. 5A performing an angioplasty procedure according to an embodiment of inventive concepts.

FIG. 6A is an illustrative perspective view of a catheter probe deploying a stent in a blood vessel according to an embodiment of inventive concepts.

FIG. 6B is an illustrative perspective view of the catheter probe of FIG. 6A analyzing the area of a vessel with a deployed stent in a blood vessel according to an embodiment of inventive concepts.

FIG. 6C is an illustrative perspective view of the catheter probe of FIG. 6A post-dilating the deployed stent of FIGS. 6A-6B according to an embodiment of inventive concepts.

FIG. 7 is an illustrative schematic of an optical source and detector configuration of a catheter according to an embodiment of inventive concepts.

FIG. 8A is an illustrative perspective view of a catheter probe with another optical configuration according to an embodiment of inventive concepts.

FIG. 8B is an expanded view of a portion of the probe tip of FIG. 8A according to an embodiment of inventive concepts.

FIG. 8C is a cross-sectional view of the probe tip portion of FIG. 8A-8B, taken along section lines I-I′ of FIG. 8B.

FIG. 9A is an illustrative side-perspective view of a tip-probe section of a catheter according to an embodiment of inventive concepts.

FIG. 9B is a perspective view of a fiber tip 45 according to an embodiment of inventive concepts.

FIG. 9C is a cross-sectional view of the fiber tip of FIG. 9B taken across section lines I-I′.

FIG. 10A is an illustrative side-perspective view of the distal end of a catheter installed within a calibration enclosure according to an embodiment of inventive concepts.

FIG. 10B is a cross-sectional view of the distal end of a catheter installed within a calibration enclosure of FIG. 10A, taken along section lines I-I′ of FIG. 10A.

### DETAILED DESCRIPTION OF EMBODIMENTS OF THE INVENTION

The accompanying drawings are described below, in which example embodiments in accordance with inventive concepts are shown. Specific structural and functional details disclosed herein are merely representative. This invention may be embodied in many alternate forms and should not be construed as limited to example embodiments set forth herein. Accordingly, specific embodiments are shown by way of example in the drawings. It should be understood, however, that there is no intent to limit the invention to the particular forms disclosed, but on the contrary, the invention is to cover all modifications, equivalents, and alternatives falling within the spirit and scope of the claims. Like numbers refer to like elements throughout the description of the figures.

It will be understood that, although the terms first, second, etc. may be used herein to describe various elements, these elements should not be limited by these terms. These terms are used to distinguish one element from another. For example, a first element could be termed a second element, and, similarly, a second element could be termed a first element, without departing from the scope of the present disclosure. As used herein, the term “and/or” includes any and all combinations of one or more of the associated listed items.

It will be understood that when an element is referred to as being “on,” “connected to” or “coupled to” another element, it can be directly on, connected to or coupled to the other element or intervening elements may be present. In contrast, when an element is referred to as being “directly on,” “directly connected to” or “directly coupled to” another element, there are no intervening elements present. Other words used to describe the relationship between elements should be interpreted in a like fashion (e.g., “between” versus “directly between,” “adjacent” versus “directly adjacent,” etc.).

The terminology used herein is for the purpose of describing particular embodiments and is not intended to be limiting of the invention. As used herein, the singular forms “a,” “an” and “the” are intended to include the plural forms as well, unless the context clearly indicates otherwise. It will be further understood that the terms “comprise,” “comprises,” “comprising,” “include,” “includes” and/or “including,” when used herein, specify the presence of stated features, integers, steps, operations, elements, and/or components, but do not preclude the presence or addition of one or more other features, integers, steps, operations, elements, components, and/or groups thereof.

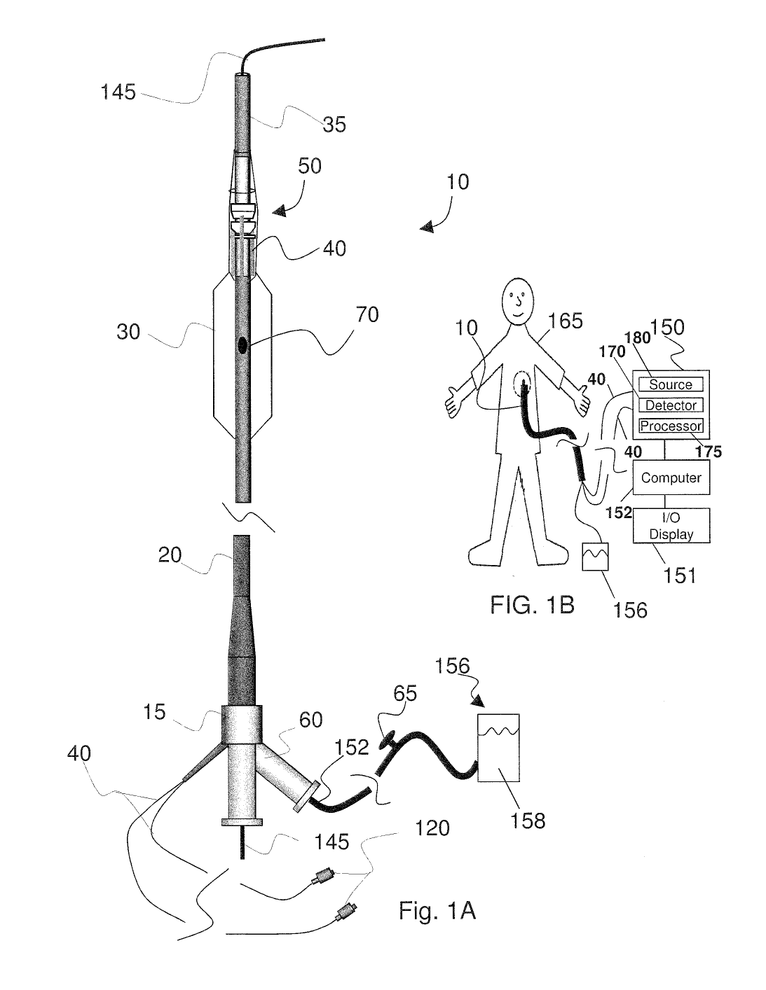

FIG. 1A is an illustrative view of a catheter instrument 10 for analyzing and medically treating a lumen, according to an embodiment of inventive concepts. FIG. 1B is a block diagram illustrating a system within an instrument 10 deployed for analyzing and medically treating the lumen of a patient with an angioplasty balloon 30, according to an embodiment of inventive concepts. The catheter assembly 10 includes a catheter sheath 20 with at least two fibers 40, including one or more delivery fiber(s) connected to at least one source 180 and one or more collection fiber(s) connected to at least one detector 170. Catheter sheath 20 includes a guidewire sheath 35 and guidewire 145. The distal end of catheter assembly 10 can optionally include a balloon 30 which, in an embodiment, can function as a lumen expanding balloon (e.g., an angioplasty balloon).

In an embodiment, a tip-probe section 50 is configured to direct illumination toward vessel walls surrounding section 50 and collect the signals returned from the vessel walls, from which the distance between section 50 and the vessel walls is measured.

The collection ends of fibers 40 are preferably configured to collect light about a wide angle such as, for example, between about at least a 120 to 180 degree cone around the circumference of each fiber, directed radially outward from about the center of catheter 10. Various methods for arranging the delivery and collection ends are described in more detail such as in related U.S. application Ser. No. 12/466,503, filed Jul. 8, 2010 and published as U.S. Patent Application Publication No. 2009/0227993 A1, the entire contents of which are incorporated herein by reference. Various such embodiments in accordance with the invention allow for diffusely reflected light to be readily delivered and collected between fibers 40 via tissue surrounding the catheter 10.

The proximate end of balloon catheter assembly 10 includes a junction 15 that distributes various conduits between catheter sheath 20 to external system components. Fibers 40 can be fitted with connectors 120 (e.g. FC/PC type) compatible for use with light sources, detectors, and/or analyzing devices such as spectrometers.

The proximate ends of fibers 40 are connected to a light source 180 and/or a detector 170 (which are shown integrated with an analyzer/processor 150). Analyzer/processor 150 can be, for example, a spectrometer which includes a processor 175 for processing/analyzing data received through fibers 40. A computer 152 with computer-readable memory is connected to analyzer/processor 150 and provides an interface for operating the instrument 10 and to further process spectroscopic data (including, for example, comparing the data to previously established model data) in order to determine the size of the lumen and/or diagnose the condition of a subject 165 for purposes of further treatment. Input/output components (I/O) and viewing components 151 are provided in order to communicate information between, for example, storage and/or network devices and the like and to allow operators to view information related to the operation of the instrument 10.

Junction 15 includes a flushing port 60 for supplying or removing fluid media (e.g., liquid/gas) 158 that can be used to expand or contract balloon 30. Fluid media 158 is held in a tank 156 from which it is pumped in or removed from the balloon(s) by actuation of a knob 65. Fluid media 158 can alternatively be pumped with the use of automated components (e.g. switches/compressors/vacuums). Solutions for expansion of the balloon are preferably non-toxic to humans (e.g. saline solution) and are substantially translucent to the selected light radiation.

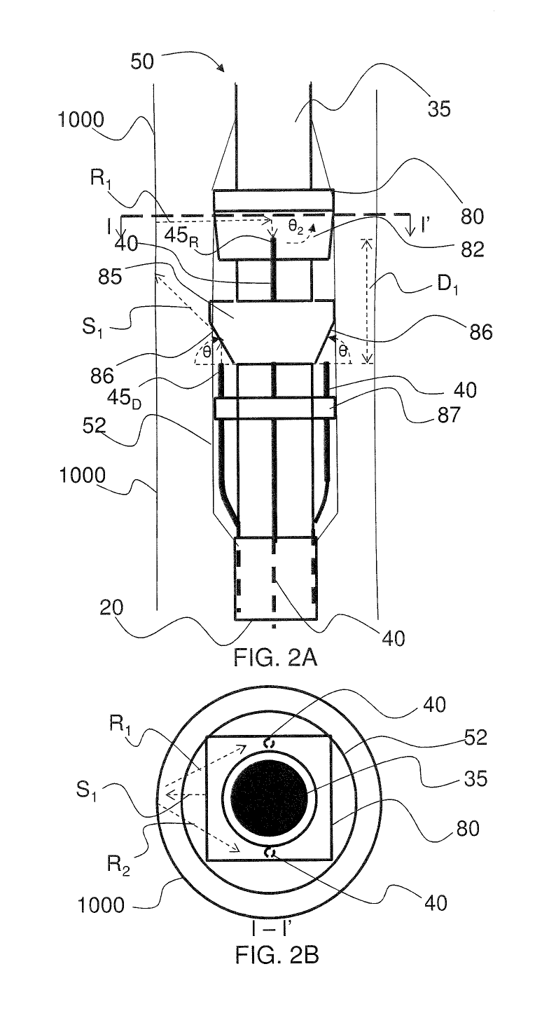

FIG. 2A is an illustrative side perspective view of a catheter tip-probe section according to an embodiment of inventive concepts. FIG. 2B is a cross-sectional view of the tip-probe section of FIG. 2A, taken along section lines I-I′ of FIG. 2A. The distal ends of fibers 40 are configured in a probe-tip arrangement 50 for delivering and collecting signals directed to and from vessel walls 1000. The probe-tip arrangement includes two reflecting elements 80 and 85 and fiber alignment segment 87. Four fibers 40 pass from within catheter sheath 35 into a translucent protective covering 52, and through alignment segment 87. Two of the fibers 40 are designated for delivering signals and have delivery tips 45_{D} that terminate adjacent to the reflective faces 86 of reflecting element 85. Two fibers 40 designated for collection pass through reflecting element 85 and have collection tips 45_{R} that terminate adjacent to reflective faces 82 of reflecting element 80. The reflective faces 86 and 82 are configured at, respectively, predetermined angles θ and θ₂ with respect to the longitudinal axis of the catheter. In various embodiments, the reflecting elements can be manufactured in a manner such as described in related U.S. patent application Ser. No. 11/834,096, published as U.S. Patent Application No. US 2007/027,0717 A1, the entire contents of which is herein incorporated by reference. Tips 45_{D} and 45_{R} are also longitudinally separated by a predetermined distance D₁. In an embodiment, θ, θ₂ and D₁ allow for an adequate return signal R₁ while avoiding undesired leakage of a signals directly between tips 45_{D} and 45_{R} (i.e., without first being reflected off lumen wall 1000). In operation of the device, a delivery signal (e.g., with exemplary path S₁) from a tip 45_{D} is reflected off of reflecting element 85 toward lumen wall 1000. In an embodiment, θ is between about 45 and 70 degrees and θ₂ is between about 45 and 70 degrees. In an embodiment, both θ and θ₂ are about 45 degrees. In another embodiment, θ is between about 65 and 70 degrees and θ₂ is about 45 degrees. In an embodiment, D₁ is between and 2 and 4 mm and preferably about 2.5 mm. A return signal (e.g., with exemplary path R₁) is collected by a collection fiber 40 with tip 45_{R} via the reflecting element 80. The delivery signal can be transmitted via a source such as described further herein. The collected signal can be analyzed such as via a spectrometer by measuring, for example, intensity and absorption of the signals through the medium between the catheter and vessel wall. As described herein, the signal measurements can be used to measure the distance between the catheter and lumen 1000 in order to determine the size and shape of the lumen 1000.

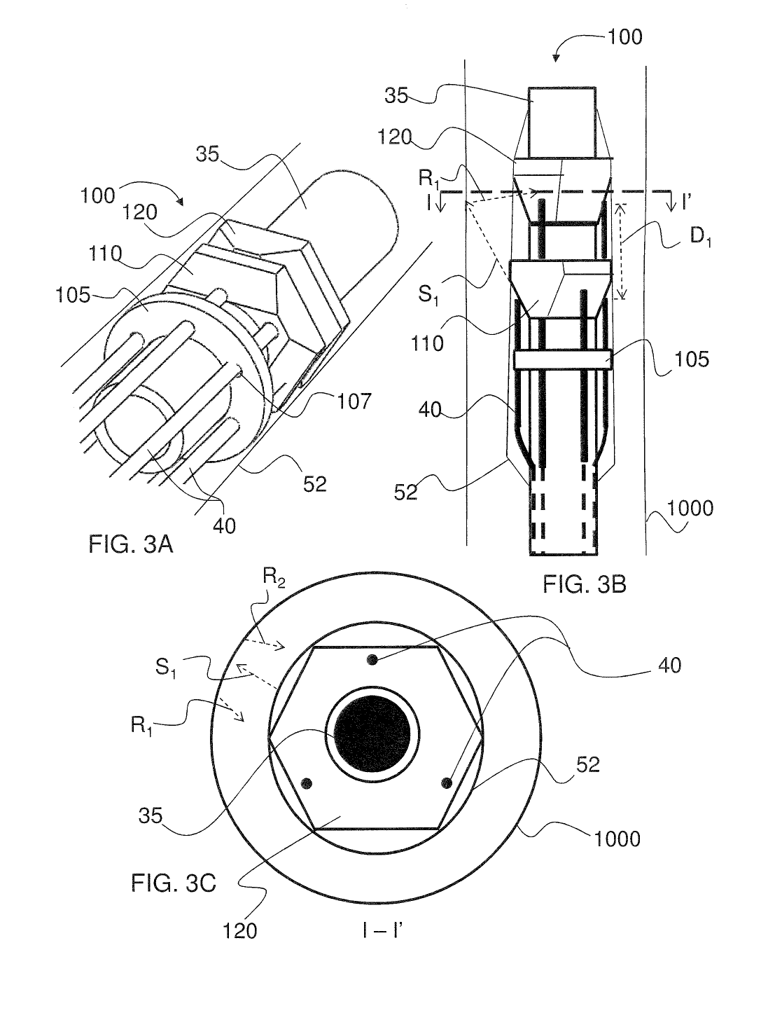

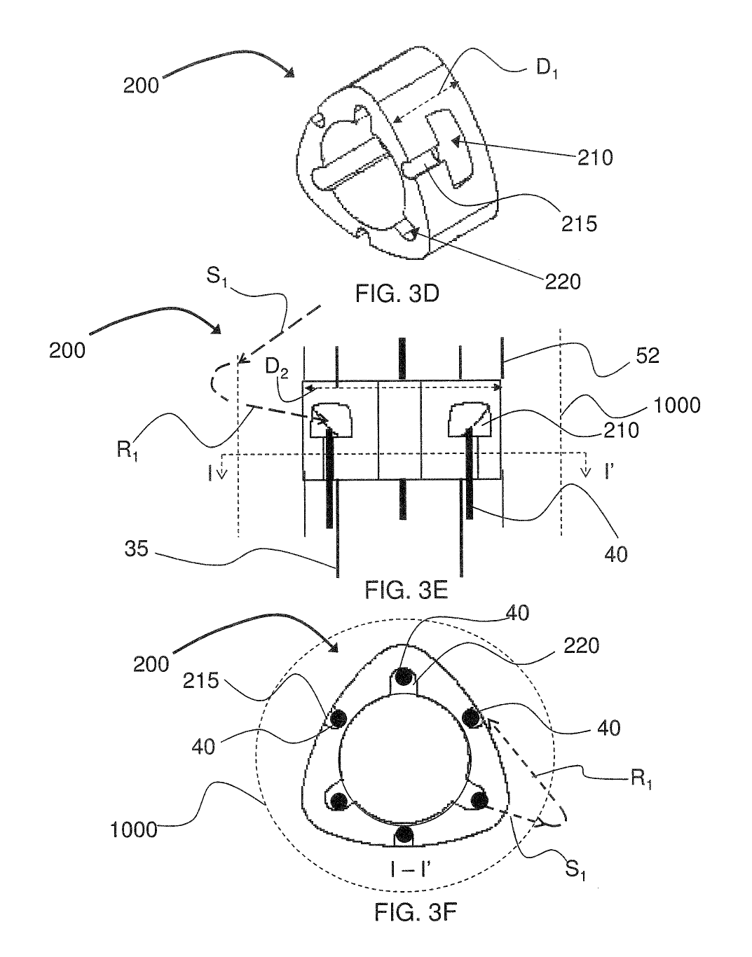

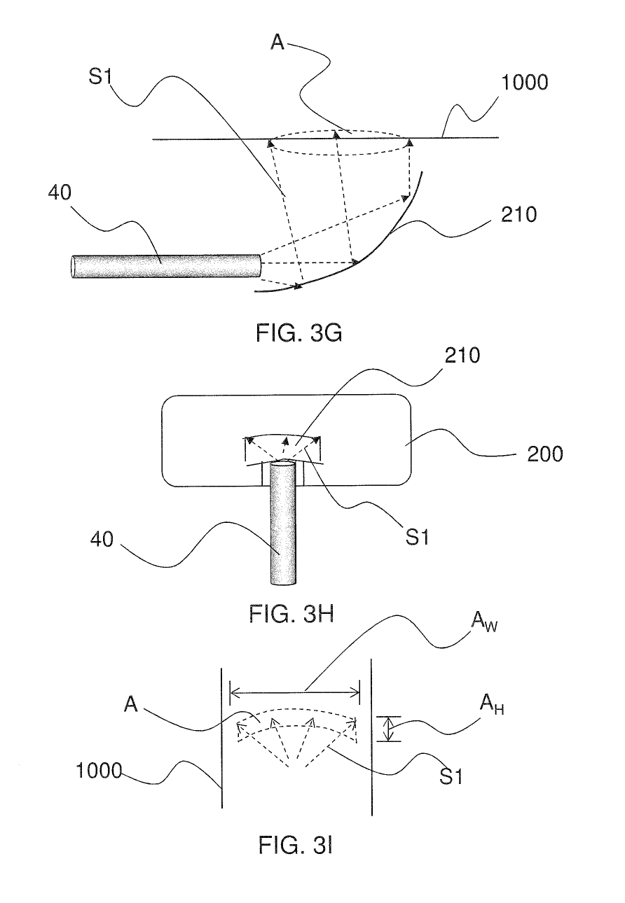

FIG. 3A is an illustrative perspective view of a tip-probe section with 6 fibers according to another embodiment of inventive concepts. FIG. 3B is an illustrative side-perspective view of the probe section of FIG. 3A. FIG. 3C is a cross-sectional view of the tip-probe section of FIG. 3A taken along section lines I-I′ of FIG. 3B. In an embodiment, a catheter 100 includes 6 fibers 40, 3 designated for delivery of signals and 3 designated for collection. Each fiber 40 passes through alignment holes 107 in an alignment segment 105. In a manner similar to each of two fibers 40 of the embodiment of FIGS. 2A-2B, three of the fibers 40 are designated for delivery and terminate at angled reflective faces of a reflective element 110 for directing signals toward the lumen wall 1000. Correspondingly, three of the fibers 40 are designated for collection and collect signals via an angled face of a reflective element 120. Embodiments of present inventive concepts can include any number of delivery and collection fibers, dependent on physical constraints relating to the particular application (e.g., lumen size, shape, flexibility). For example, peripheral, aortal, or other large vessels can have diameters up to about 30 mm or more and could accommodate, for example, a 20 plus fiber catheter according to inventive concepts. In an embodiment, a catheter according to present inventive concepts can be used in connection with a transcatheter aortic valve implantation (TAVI) procedure. As shown in FIG. 3C, an exemplary signal travels via a path S₁ and signals are then collected via collection fibers 40 circumferentially adjacent (or 60 degrees separated) from the delivery fiber tip (e.g., along exemplary paths R₁ and R₂). Six or more can lead to a larger footprint (e.g., overall catheter diameter) but can provide greater detail relating to features of the surrounding lumen than, for example, 4 fibers. Also, the non-circular shape of the segments 105, 110, and 120 of FIGS. 3A-3C, as are segments 80, 85, and 87 of FIGS. 2A-2B, allows for more accurate rotational alignment between the separated segments.

FIG. 3D is an illustrative perspective view of an alignment and reflector segment 200 according to an embodiment of inventive concepts. FIG. 3E is a side perspective view of the alignment and reflector segment 200 of FIG. 3D. FIG. 3F is a cross-sectional view of the alignment and reflector segment 200 of FIG. 3D, taken along section lines I-I′ of FIG. 3E. The segment 200 includes an alignment groove for aligning the tip of fiber 40 with a reflector 210. The segment 200, including the alignment grooves 215, are enclosed within a translucent protective sheath 52, which retains fibers 40 in the grooves 215. Additional fibers 40 pass through inner grooves 220, such as to another alignment reflecting element, and are retained against a guidewire lumen 35. Additional open space within grooves 220, permitting more play of fibers 40, allows some movement of fibers 40 as the catheter is bent and turned during deployment. The depth of grooves 220 can be, for example, between 0.1 and 0.2 mm while the depth of grooves 215 can be about half that depth. The maximum length D₁ of the segment can be between approximately 0.4 to 0.5 mm for a coronary targeted application. The maximum diameter D₂ can be between approximately 0.8 and 1 mm for a coronary targeted application. Referring to FIG. 3E, a sample delivery signal S₁ is traced toward a surrounding lumen 1000, then returned along a sample trace R₁ toward an angled reflective facet 210 and then received through the tip of a fiber 40. The isolation and smaller relative size of reflective facets 210, as opposed to the reflective faces of FIGS. 2A-3C, can help decrease leakage of direct signal between delivery and collection fiber tips.

FIG. 3G is a side perspective view of a fiber and curve-shaped reflector 210 according to an embodiment of inventive concepts. FIG. 3H is another side-perspective view of a reflector 210 incorporated into a multi-faceted reflective piece 200 according to an embodiment of inventive concepts. FIG. 3I is a side perspective view of a lumen illuminated by the fiber and reflector 210 of FIGS. 3G and 3H. As well as being flat-shaped such as illustrated in FIGS. 2A-2B and 3A-3C, reflective facets such as facets 210 can also have curved surfaces such as for concentrating light delivered to or collected from a particular region of interest (ROI) such as A. Facets 210 can also be dimensioned to shape the ROI to a more rectangular shape, reflective of facet 210 in FIG. 3H, and an ROI A with dimensions A_{W} and A_{H}. Because the primary direction of transmission or collection of longitudinally pointed fiber tips is in the longitudinal direction, distribution of a higher proportion of the ROI in the circumferential direction can help avoid direct transmission (cross-talk) between delivery and collection fibers that are longitudinally separated such as shown in, for example, FIGS. 2A and 3B. This feature also helps promote a more circumferentially expansive and evenly distributed ROI profile of the lumen wall.

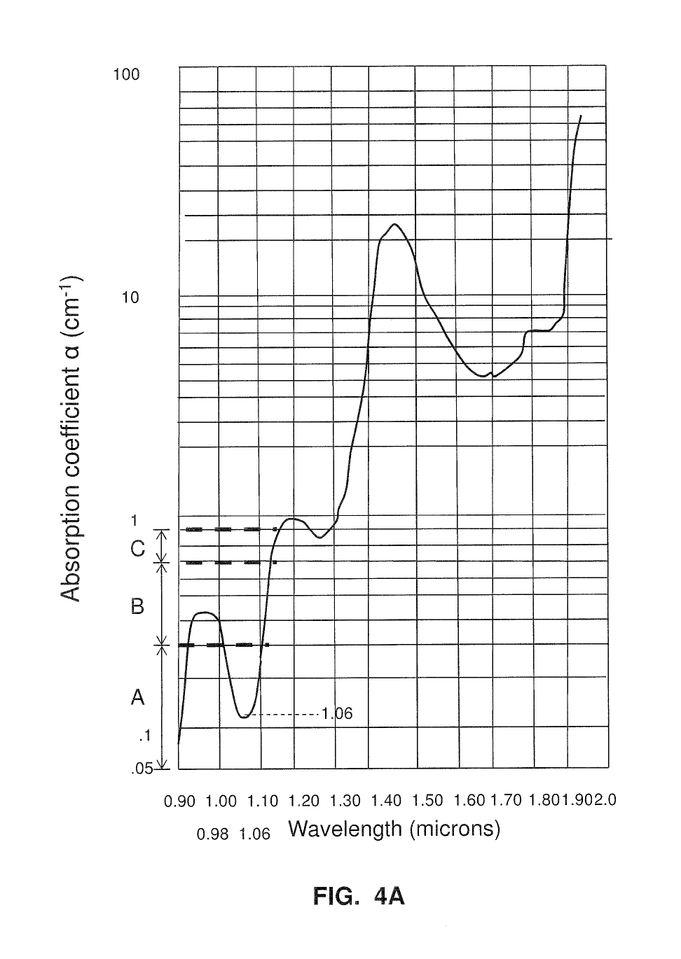

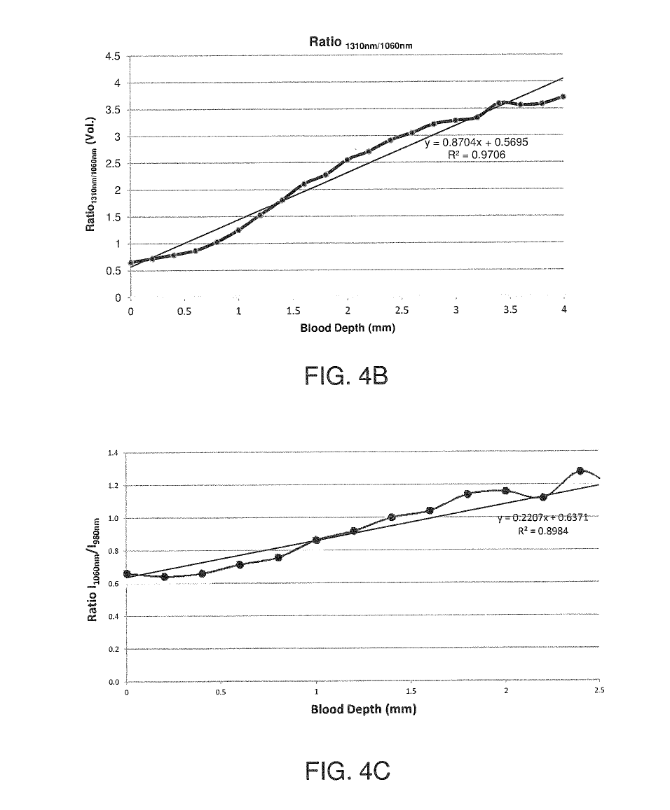

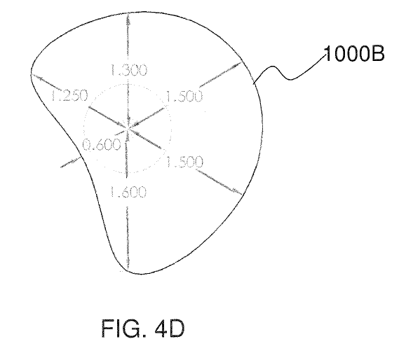

FIG. 4A is a logarithmic chart of measured absorption coefficients in water relative to selected near-infrared wavelengths of light. In an embodiment of inventive concepts, near-infrared signals are ideal for traveling through an aqueous media such as blood because of their absorbance and low scattering properties such as described in related U.S. patent application Ser. No. 12/784,482, published as U.S. Patent Application Publication No. US 2010-0286531 A1, the entire contents of which is herein incorporated by reference. In addition to being absorbed by the water content within blood, the signals are also absorbed by other blood components (e.g., hemoglobin) and tissue of the adjacent lumen and thus a minimal absorbance rate is desirable for distance measurements made through blood against tissue. In an embodiment for measuring a distance between the catheter probe and lumen wall, an optimal wavelength provides a measurable intensity difference through blood and reflects well off the lumen wall. In addition, the reflectivity of the optimal wavelength is not significantly affected by the content of the lumen such as, for example, collagen, cholesterol, and/or plaque which are present in vessels to varying degrees and can significantly change the reflectivity of other wavelengths, including wavelengths, for example, of about 1200 and 1389 nm.

FIG. 4B is a chart of intensity measurement ratios measured through bovine blood at varying distances between a bovine blood vessel wall and catheter wall using wavelengths of 1310 nm and 1060 nm. In an embodiment, a reading wavelength (also referred to herein as a primary wavelength) is selected so as to provide a scattering-based measurement of the distance from the catheter to the lumen wall, that is, the reading wavelength scatters in a predictable manner when passing through a blood medium across a span of about 3 mm or less. As a lumen wall increases in distance from the separated probe ends, the level of scattering in the adjacent blood increases and the amount absorbed by the tissue decreases, thereby increasing the overall signal returned back to the probe. In an embodiment, a scattering reading wavelength of between about 1030 nm and 1100 nm, such as range B shown in FIG. 4A, is used, and preferably a wavelength of about 1060 nm. FIG. 4B demonstrates an example of using a scattering (reading) wavelength to measure distance. Lower wavelengths can be excessively absorbed by the hemoglobin content and greater wavelengths excessively absorbed by the water content. A lumen wall is highly absorbent of wavelengths in this range. As a source of this wavelength range (e.g., a fiber tip) approaches a lumen wall, a significantly greater component of the signal will be absorbed by the wall. As the source of the emission moves away from the wall, less light is absorbed by the wall and less light overall is absorbed, much of it reflecting and scattering back to the source. In an embodiment, an optimal reference wavelength does not appreciably change in scattering across a span of about 3 mm or less and has similar absorbance in blood and reflectivity from the lumen wall as the reading wavelength. In an embodiment, a reference wavelength is between about 1250 nm and 1400 nm and preferably a wavelength of about 1310 nm. In an embodiment, the measured intensity of the primary wavelength is divided into the measured intensity of the reference wavelength to provide a normalized ratio such as shown in FIG. 4B. In an embodiment, near-infrared diffuse reflectance spectroscopy is employed. Other manners of spectroscopy including, for example, Raman spectroscopy, fluorescence spectroscopy, optical coherence reflectometery, and optical coherence tomography, can also be utilized.

FIG. 4C is a chart of absorption measurement ratios measured through bovine blood at varying distances between a bovine blood vessel wall and catheter wall using wavelengths of 980 nm and 1060 nm. In an embodiment, a reading wavelength is selected so as to provide a reflectance-based measurement of the distance from the catheter to the lumen wall, that is, the reading wavelength diffusely reflects in a predictable manner when passing through a blood medium and reflecting off the lumen wall. In an embodiment of inventive concepts, an optimal primary reflectance wavelength (or reading wavelength) for transmission through blood of a distance of 4 mm or less to an adjacent lumen wall and back has a wavelength with an absorption coefficient (cm⁻¹) between about 0.05 and 0.3 or between about 0.7 and about 1 (e.g., as shown as regions A and C of FIG. 4A). In an embodiment, one or more primary/reading wavelengths are between about 900 and 1000 nanometers and/or between about 1120 and 1150 nanometers. In order to account for background effects that impact the primary signal, a reference signal with little change in absorbance over the span (of about 4 mm) can be measured through the same medium and compared to the primary signal. In an embodiment, an absorbance reference wavelength with relatively low reflectivity against a lumen wall compared to the primary wavelength can be used to help normalize the signal of the reading wavelength. In an embodiment, the absorbance reference wavelength has an absorption coefficient (cm⁻¹) between about 0.3 and 0.7 (as shown as region B of FIG. 4A). In an embodiment, the absorbance reference wavelength is between about 1020 and 1120 nm.

FIG. 4B is a chart of absorption measurement ratios measured ex-vivo within a harvested bovine coronary artery with bovine blood mechanically pumped through it. Measurements were taken with fibers circumferentially separated by 90 degrees (such as in a 4 fiber arrangement as described above in reference to FIGS. 2A-2B). Absorption readings were taken at distances across 0 to 4 mm between a collection fiber tip and vessel wall using lasers with outputs of 1310 and 1060 nm. The distances through which the readings were taken were independently verified using a high-precision micrometer stage and sensor for determining if the probe tip and vessel were in contact. As shown in the chart, the relationship between the ratio of absorption coefficients is relatively linear. The relationship for a particular configuration/tip probe arrangement can be similarly studied in human subjects and programmed into the computer-readable memory of a console/controller (e.g., computer 152 of FIG. 1B), and compared to measurements taken in patients for purposes of treatment. In further embodiments of inventive concepts, a multivariate approach of analysis can be performed in relation to multiple pre-configuration measurements taken in test settings. In an embodiment, the methods include multiple regression analysis, logistic regression analysis, discriminant analysis, multivariate analysis of variance, factor analysis, cluster analysis, multidimensional scaling, correspondence analysis, conjoint analysis, canonical correlation, and structural equation modeling and other general analytical methods known to those of ordinary skill in the art.

FIG. 4D is an illustrative chart representing calculations made from exemplary distance measurements according to embodiments of the invention. The calculations represent measurements that could be taken from an embodiment with fibers separated by 60 degrees (such as in a 6 fiber arrangement as described above in reference to FIGS. 3A-3C). The exemplary signals convey a generally normally shaped/sized lumen shape 1000B except in a lower left portion, where exemplary signals indicate a significant narrowing of the lumen. Depending on the context of the measurements (e.g., pre-dilitation, post-stenting), the calculations can provide an assessment of the size (e.g., area and volume) and shape of a diseased blood vessel, and/or identify an under-expanded or mal-apposed stent in the area being analyzed. The shape of the lumen can be estimated from the distance measurements using, for example, spline or other fitting techniques known to those of skill in the art.

In an embodiment, the analysis of the lumen wall further includes information which can be spectroscopically analyzed to measure certain characteristics such as a change of chemical components, tissue morphological structures, water/blood content, and physiological parameters (e.g. temperature, pH, color, intensity) in the lumen wall. These components include the identification of plaque, collagen content, lipid content, calcium content, inflammatory factors, and the relative positioning of these features within the plaque. Absorption of wavelengths in the near-IR spectrum are known to measurably change in the presence of these components (see, e.g., U.S. Patent Application Publication No. US 20070078500 A1 by Ryan et al., U.S. Patent Application Publication No. 2004/0111016 A1 by Casscells, III et al., and U.S. Pat. No. 7,486,985 by Marshik-Geurts et al., the entire contents of each of which is herein incorporated by reference). In an embodiment, as few as two wavelengths in the near-IR spectrum are needed to measure these properties such as described in U.S. Pat. No. 7,486,985, referenced above. In some ranges and wavelengths, including those less than 1100 nm and some greater than 1415 nm, the distance of the probe input/outputs from the lumen wall significantly affect any signal association with, for example, plaque content, which precludes the availability of these wavelengths for tissue content analysis without an additional distance reference. In an embodiment, the above described methods of distance measurement are used to qualify signals collected to assess at least one of chemical components and tissue morphological structures. In an embodiment, at least one signal of less than about 1100 nm or between about 1415 and 1500 nm is analyzed to assess the presence of plaque, chemical components, tissue morphological structures, water content, blood content, temperature, pH, and/or color. In an embodiment, two or less signals, one of which is less than about 1100 nm or between about 1415 and 1500 nm, is analyzed for such assessment. In an embodiment, a distance reading wavelength and distance reference wavelength are analyzed as described above and combined with a signal of less than 1100 nm or between about 1415 and 1500 nm which is, combined with the measured distance, used to identify and/or measure at least one of the above described chemical/physiological parameters.

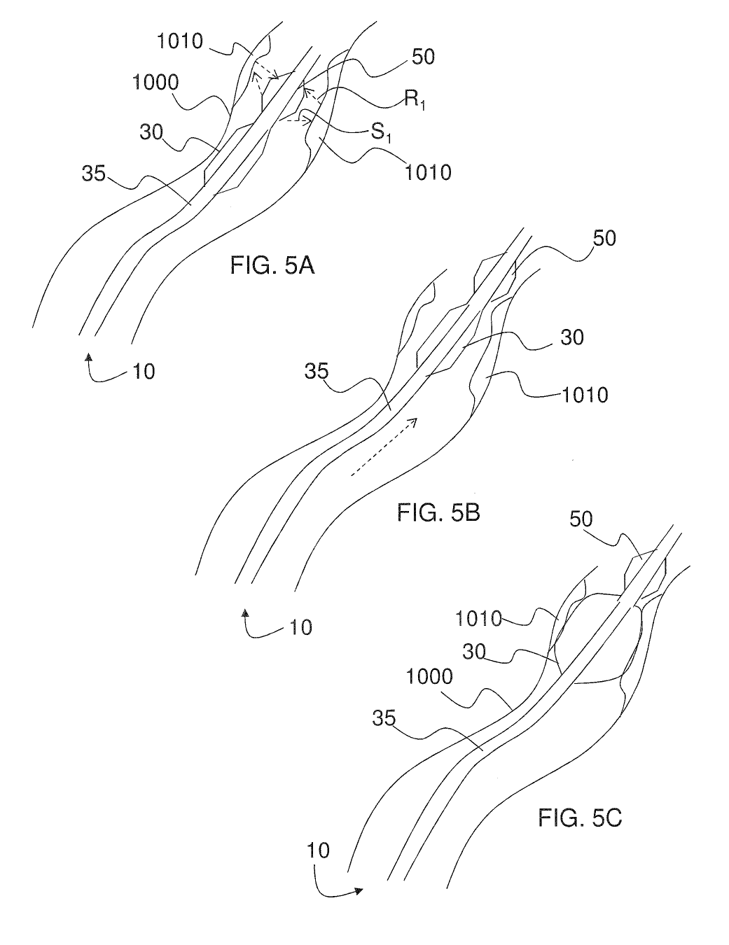

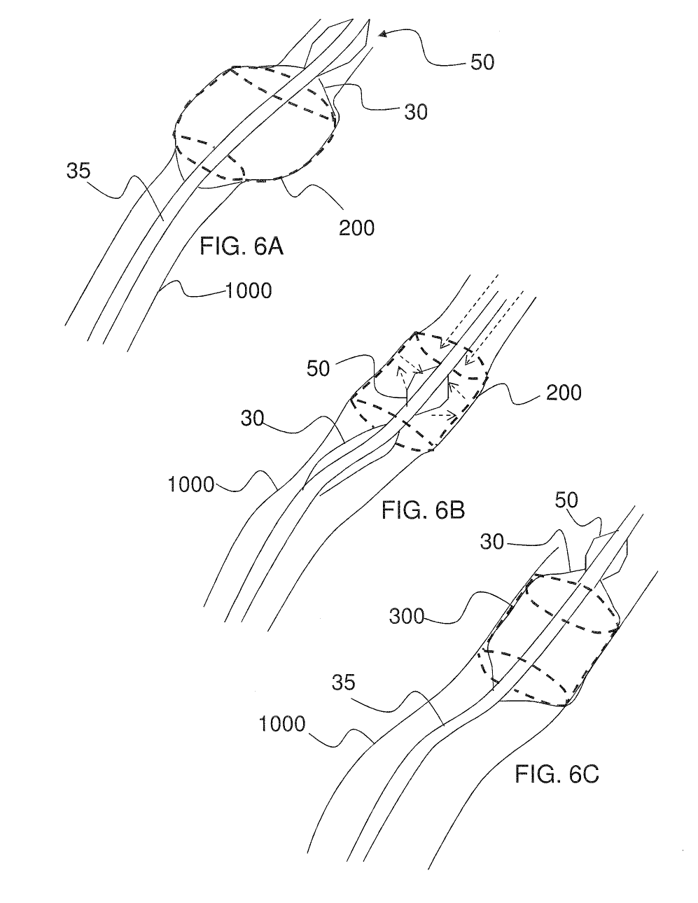

FIG. 5A is an illustrative perspective view of a catheter probe deployed within and analyzing a blood vessel according to an embodiment of inventive concepts. FIG. 5B is an illustrative perspective view of the catheter probe of FIG. 5A positioned for an angioplasty procedure according to an embodiment of inventive concepts. FIG. 5C is an illustrative perspective view of the catheter probe of FIG. 5A performing an angioplasty procedure according to an embodiment of inventive concepts. As shown in FIG. 5A, a catheter 10 according an embodiment of inventive concepts can be first positioned in a lumen 1010 so that a probe segment 50 is positioned adjacent to constricted lumen area 1010. Catheter 10 additionally includes a catheter sheath 35 with an angioplasty balloon 30 such as within various embodiments described herein. Analysis of the constricted lumen area 1010 according to an embodiment of inventive concepts can indicate the size, including length, and shape of the lumen such as for determining what size of a stent is needed and how large the stent should be expanded within the lumen. Either with a stent (not shown) in place or for pre-dilitation (without a stent) of the lumen 1000, angioplasty balloon 30 can then be positioned into place (e.g., see FIG. 5B) within constricted area 1010 so that the area 1010 can be expanded (e.g., see FIG. 5C) and/or stented with the use of information gained through analysis with probe segment 50. The lumen area 1010 can then be further analyzed by moving probe segment 50 back into place and adjacent to lumen area 1010 such as shown in FIG. 5A and FIG. 6B.

FIG. 6A is an illustrative perspective view of a catheter probe deploying a stent 300 in a blood vessel 1000 according to an embodiment of inventive concepts. FIG. 6B is an illustrative perspective view of the catheter probe of FIG. 6A analyzing the area of a vessel with a deployed stent in a blood vessel according to an embodiment of inventive concepts. FIG. 6C is an illustrative perspective view of the catheter probe of FIG. 6A post-dilating the deployed stent of FIGS. 6A-6B according to an embodiment of inventive concepts. Once the probe section 50 is in place within stent 300 (as shown in FIG. 6B), the stented area can be examined through probe section 50. The measured size of the lumen can indicate whether the stent is under-expanded. A profile as exemplified by FIG. 4B, for example, could indicate a mal-apposed stent. In an embodiment, the stenting procedure can be accomplished with a self-expanding stent such as those, for example, made of nitinol having a pre-shaped memory profile.

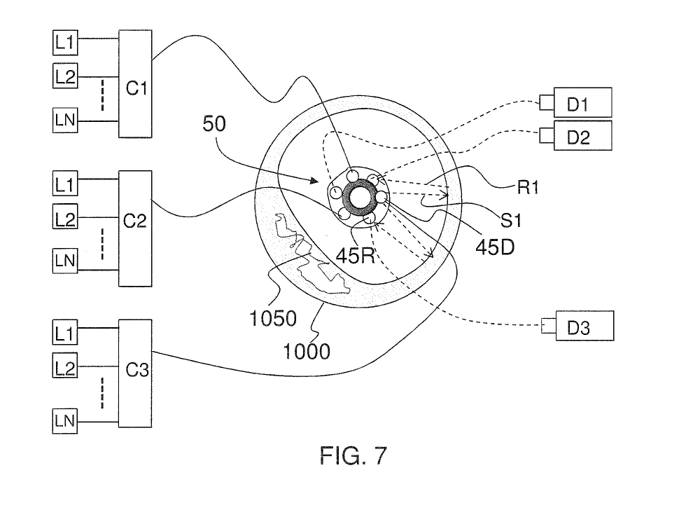

FIG. 7 is an illustrative schematic of an optical source and detector configuration of a catheter according to an embodiment of inventive concepts. In the embodiment shown, three channels, C1, C2, and C3, are designated for delivering light to 3 corresponding delivery fiber tips 45D. Each of the channels are connected to multiple light sources (e.g., lasers L1, L2, . . . LN) that can either be combined or switched on/off by a controller (not shown). Each of 3 collection fiber tips 45R is connected to a detector, D1, D2, and D3. An exemplary signal S1 is delivered to the wall of the lumen 1000 through channel C3 and an exemplary signal R1 is received by detector D2.

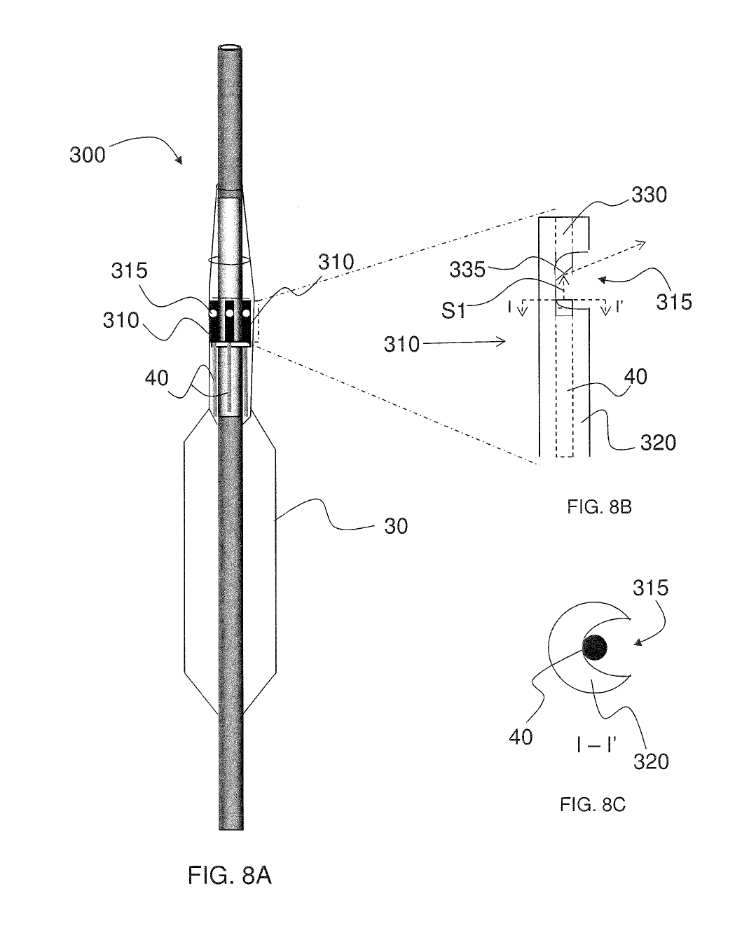

FIG. 8A is an illustrative perspective view of a catheter probe tip 300 with another optical configuration according to an embodiment of inventive concepts. FIG. 8B is an expanded view of a portion of the probe tip of FIG. 8A according to an embodiment of inventive concepts. FIG. 8C is a cross-sectional view of the probe tip portion of FIG. 8A-8B, taken along section lines I-I′ of FIG. 8B. In an embodiment, the probe tip includes a section 310 at the distal end of each fiber. Section 310 includes a tubular segment 320 with a hole through which a fiber 40 passes and through which passes a wire 330 having a reflective surface 335 positioned opposite to the exposed end of fiber 40. Tubular segment 320 also includes an open area 315 that allows light to travel to and from the exposed ends of fiber 40 and wire 330. The reflective surface 335 allows light to travel obliquely relative to the longitudinal axis of fiber 40 and be delivered or collected by fiber 40 (e.g., exemplary signal path S1). The reflective surface 335 of wire 330 can be cleaved or shaped at a predetermined angle such as described above in reference to angles θ and θ₂. In an embodiment, wire 330 can be made of steel, aluminum, or copper or other suitable material. In an embodiment, the reflective face 335 of the wire is coated with a reflective material such as gold. The coating can be applied, for example, by ion-assisted deposition. In an embodiment, the tubular segment is a molded plastic piece.

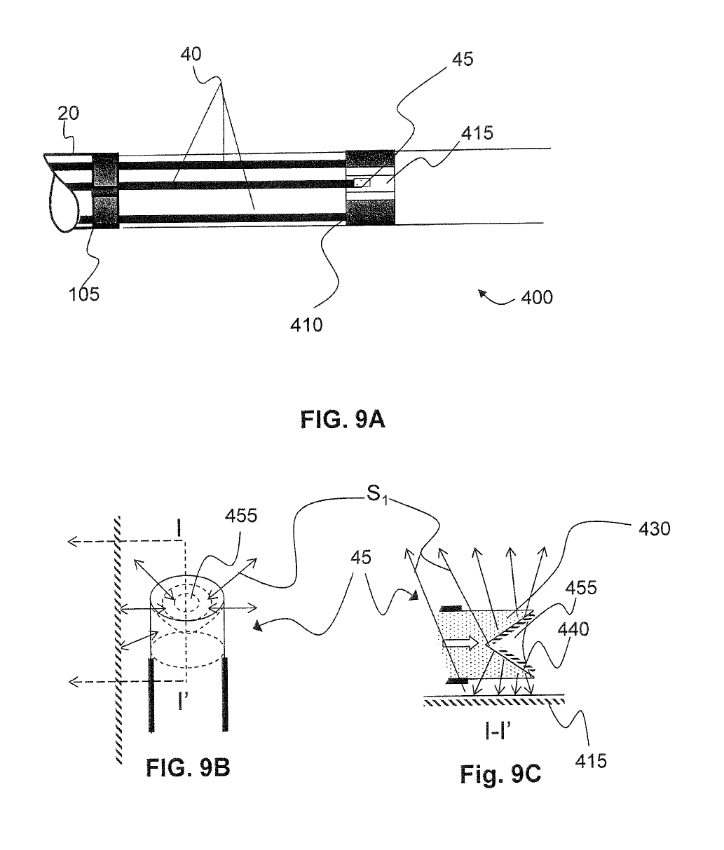

FIG. 9A is an illustrative side-perspective view of a tip-probe section 400 of a catheter according to an embodiment of inventive concepts. FIG. 9B is a perspective view of a fiber tip 45 according to an embodiment of inventive concepts. FIG. 9C is a cross-sectional view of the fiber tip 45 of FIG. 9B taken across section lines I-I′. In an embodiment, a fiber tip 45 is constructed so as to distribute or collect light to or from a direction transverse to the direction of the fiber's longitudinal axis. In an embodiment, the tip 45 is constructed having a core 430 in which a recess 455 is located at its terminating end. In an embodiment, the core can have a depth of less than about 70 microns and, in an embodiment, between about 50 and 70 microns. Such tips can be constructed using an etching process such as described in U.S. Patent Application Publication No. US 20090227993 A1 entitled “SHAPED FIBER ENDS AND METHODS OF MAKING SAME,” the entirety of which is incorporated herein by reference. A coating 440 can cover the recess and aid in re-directing light traveling to or from core 430 such as exemplified by signals S₁. A reflecting element 410, including a reflective face 415, can aid in directing or collecting light to or from a direction outward from the catheter tip 400.

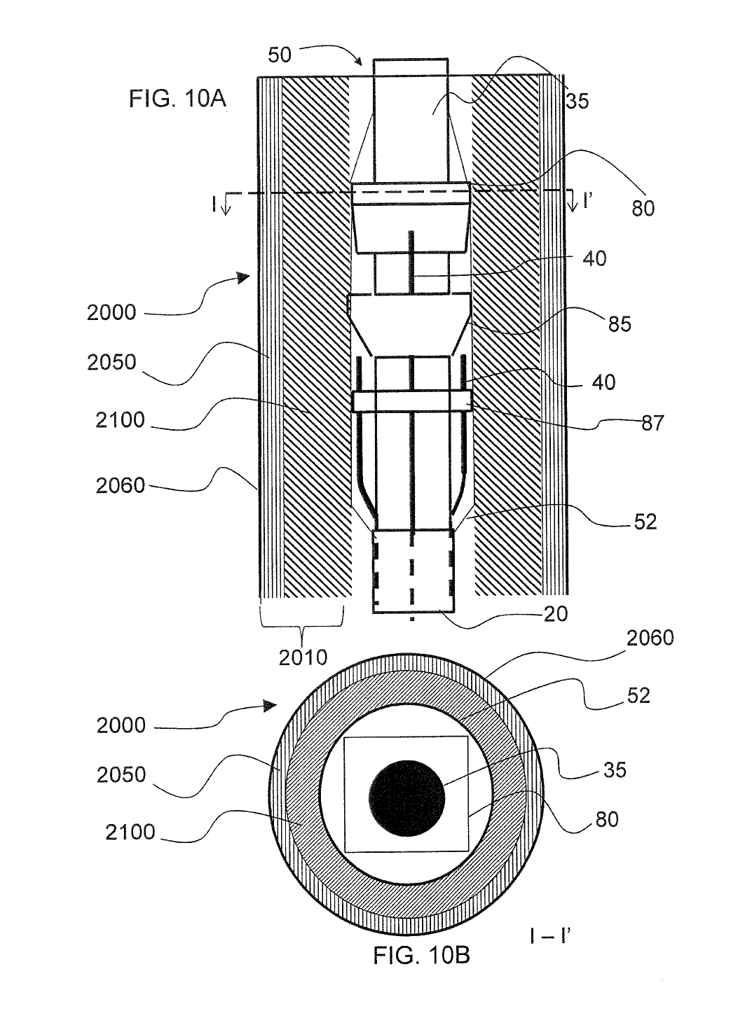

FIG. 10A is an illustrative side-perspective view of the distal end of a catheter installed within a calibration enclosure 2000 according to an embodiment of inventive concepts. FIG. 10B is a cross-sectional view of the distal end of a catheter installed within a calibration enclosure 2000 of FIG. 10A, taken along section lines I-I′ of FIG. 10A. In an embodiment, prior to analysis of a live subject, embodiments of catheters such as represented herein (e.g., of FIGS. 2A-2B) can undergo a pre-deployment calibration in a calibration enclosure 2000 wherein signals from the catheter system are distributed and collected by the catheter within the enclosure and analyzed in order to correct/adjust for future measurement calculations made when deployed within the lumen of a live subject. In an embodiment, the enclosure includes a tissue-phantom 2010 which interacts with signals from the catheter in a predictable manner and, through the analysis of signals delivered/received through the phantom, provides correlation parameters which can later be applied to actual tissue/blood measurements in order to optimize distance calculations. In an embodiment, the tissue phantom 2000 includes a blood-simulation component 2100 and a tissue/lumen wall-simulation component 2050 that is held within a sheath 2060. Materials which can provide tissue/blood phantom simulation properties for the desired wavelengths include, for example, such commercially available products as provided by INO Biomimic® (based out of Quebec City, Quebec, see http://www.ino.ca/). Such calibration can help mitigate variations within the optical components among catheters that can result during manufacture and transportation.

It will be understood by those with knowledge in related fields that uses of alternate or varied materials and modifications to the methods disclosed are apparent. This disclosure is intended to cover these and other variations, uses, or other departures from the specific embodiments as come within the art to which the invention pertains.

1. A system for analyzing a body lumen comprising:
a catheter comprising a flexible conduit that is elongated along a longitudinal axis, the flexible conduit having a proximal end and a distal end;
at least one delivery waveguide and at least one collection waveguide extending along the flexible conduit, a transmission output of the at least one delivery waveguide and a transmission input of the at least one collection waveguide located along a distal portion of the conduit;
a spectrometer connected to the at least one delivery waveguide and the at least one collection waveguide, the spectrometer configured to perform diffuse reflectance spectroscopy through blood, wherein the spectrometer emits at least one primary radiation signal of a wavelength of between about 750 and 2500 nm that is directed through the transmission output to a wall of the body lumen, and wherein the transmission input collects radiation directed from the body lumen wall; and
a controller system comprising computer-readable memory programmed to store the signal measured by the spectrometer and to enable the controller to calculate a distance between the catheter and the wall of the body lumen based on a signal measured by the spectrometer of the at least one primary radiation signal that traveled through blood between the catheter and body lumen, the controller further programmed to store the calculated distance in the computer-readable memory.

2. The system of claim 1 wherein the spectrometer is further configured to perform spectroscopy of at least one reference radiation signal, and wherein the controller system is further programmed to calculate and store in the computer-readable memory a ratio of detected signals between the detected signal of the at least one primary radiation signal and a detected signal of the at least one reference radiation signal measured through blood by the spectrometer in order to calculate the distance between the flexible conduit and the wall of the body lumen.

3. The system of claim 2 wherein the at least one reference radiation signal comprises a wavelength having an absorption coefficient in water of less than about 8 cm⁻¹.

4. The system of claim 3 wherein the at least one reference radiation signal comprises a wavelength having an absorption coefficient in water of between about 0.3 and 0.7 cm⁻¹.

5. The system of claim 4 wherein the at least one reference radiation signal comprises a wavelength of between about 1020 and 1120 nm.

6. The system of claim 3 wherein the at least one reference radiation signal comprises a wavelength of about 1060 nm.

7. The system of claim 2 wherein the at least one reference radiation signal comprises a wavelength of about 1310 nm.

8. The system of claim 7 wherein the at least one primary radiation signal comprises a wavelength of about 1060 nm.

9. The system of claim 2 wherein the computer-readable memory is programmed with an algorithm for enabling the controller to calculate a ratio of detected signals between the detected signal of the at least one primary radiation signal and an detected signal of at least one reference radiation signal and comparing the ratio to previously calculated and stored ratios measured from one or more catheters correspondingly configured to said catheter comprising a flexible conduit.

10. The system of claim 1 wherein the at least one delivery waveguide and at least one collection waveguide are arranged to measure the at least one primary radiation signal across a plurality of regions distributed about the circumference of the conduit and between the flexible conduit and the wall of the body lumen.

11. The system of claim 10 wherein the computer-readable memory is programmed to enable the controller to calculate a cross-sectional area of the lumen from the measurements across the plurality of regions.

12. The system of claim 1 wherein the at least one primary radiation signal comprises a wavelength having an absorption coefficient in water of between about 0.05 and 0.3 cm⁻¹.

13. The system of claim 12 wherein the at least one primary radiation signal comprises a wavelength between about 900 and 1000 nm.

14. The system of claim 1 wherein the at least one primary radiation signal comprises a wavelength having an absorption coefficient in water of between about 0.7 and 1 cm⁻¹.

15. The system of claim 14 wherein the at least one primary radiation signal comprises a wavelength between about 1120 and 1150 nm.

16. The system of claim 1 wherein the at least one primary radiation signal comprises a wavelength having an absorption coefficient in water of between about 0.3 and 0.7 cm⁻¹.

17. The system of claim 16 wherein the at least one primary radiation signal comprises a wavelength of between about 1020 and 1120 nm.

18. The system of claim 1 wherein the computer-readable memory is programmed with an algorithm that represents a multivariate analysis of preliminary measurements taken from one or more catheters correspondingly configured as said catheter comprising a flexible conduit.

19. The system of claim 18 wherein the multivariate analysis comprises at least one of multiple regression analysis, logistic regression analysis, discriminant analysis, multivariate analysis of variance, factor analysis, cluster analysis, multidimensional scaling, correspondence analysis, conjoint analysis, canonical correlation, and structural equation modeling.

20. The system of claim 1 wherein the catheter further comprises a removable calibration sheath surrounding the transmission output of the at least one delivery waveguide and the transmission input of the at least one collection waveguide, the calibration sheath arranged to return radiation to the transmission input of the at least one collection waveguide in response to receiving radiation from the transmission output of the at least one delivery waveguide.

21. The system of claim 20 wherein the calibration sheath comprises a tissue phantom so as to permit simulation of delivering radiation from the transmission output to the tissue phantom and receiving radiation from the tissue phantom through the transmission input.

22. The system of claim 21 wherein the tissue phantom comprises at least one of an artificial blood phantom and artificial blood vessel wall phantom.

23. The system of claim 20 wherein the calibration sheath is arranged to improve the accuracy of the calculation of a distance between the catheter and the wall of the body lumen by the calculation of calibration factors that are programmed to be calculated by the controller and stored in computer-readable memory after operating the spectrometer with the calibration sheath in place over the catheter.

24. The system of claim 1 further comprising an angioplasty balloon disposed about a distal portion of the conduit.

25. The system of claim 24 wherein the transmission output of the at least one delivery waveguide and the transmission input of the at least one collection waveguide is located within the angioplasty balloon.

26. The system of claim 1 wherein the at least one delivery waveguide and collection waveguide comprises a fiber optic that has an end that operates as a reflection surface for changing a direction of a path of radiation to or from a direction transverse to the axis of the fiber optic.

27. The system of claim 26 wherein the end of the fiber optic comprises a tip with
a core and a recess formed in said core at a distal end of the optical fiber tip to direct radiation transversely from the longitudinal axis of the fiber optic, said recess having a vertex within said core and the core having a maximum depth of less than about 70 microns.

28. The system of claim 26 further comprising a first optical element disposed about the flexible conduit, the optical element including an array of multiple facets that lie at an acute angle relative to the longitudinal axis of the flexible conduit for changing a direction of radiation transmitted to or from a longitudinal axis of the at least one delivery or collection waveguide so that the radiation is emitted or collected to or from a direction that is transverse to the longitudinal axis of the at least one delivery or collection waveguide.

29. The system of claim 28 wherein at least one of the multiple facets comprises a width along the circumference of the flexible conduit that is at least 1.5 times the height of the at least one facet along the longitudinal direction of the flexible conduit.

30. The system of claim 28 wherein the at least one of the multiple facets comprises the shape of a concave parabola so as to further concentrate the delivery or collection of a signal across a longitudinal span of the lumen wall.

31. The system of claim 28 further comprising a second optical element for aligning distal ends of the at least one delivery or collection waveguide with the reflective facets of the first optical element.

32. The system of claim 28 wherein the second optical element segment includes at least one feature for aligning the distal ends of the at least one delivery or collection waveguide with the reflective facets.

33. The system of claim 32 wherein said at least one feature includes a shape having a plurality of flat sides arranged about the circumference of the conduit so as to rotationally align with the reflective facets.

34. The system of claim 31 wherein the second optical element comprises at least one of holes or grooves extending along the entire longitudinal extent of the second optical element through which at least one of the at least one delivery waveguide and collection waveguide passes through.

35. The system of claim 34 wherein the second optical element further comprises an array of multiple facets that lie at an acute angle relative to the longitudinal axis of the flexible conduit for changing a direction of radiation transmitted to or from a longitudinal axis of at least one delivery or collection waveguide so that the radiation is emitted or collected to or from a direction that is transverse to the longitudinal axis of the at least one delivery or collection waveguide.

36. The system of claim 35 wherein the facets of the first optical element are separated from the facets of the second optical element by a predetermined longitudinal distance.

37. The system of claim 36 wherein the predetermined longitudinal distance is about 2.5 mm.

38. The system of claim 31 wherein at least one of the first and second optical elements is configured for delivering signals to an adjacent lumen and at least one of the first and second optical elements is configured for collecting signals from the adjacent lumen.

39. The system of claim 31 wherein at least one of the waveguides terminates at one of the multiple facets.

40. The system of claim 1 wherein the computer-readable memory of the controller is further programmed to enable the controller to measure at least one of the characteristics of plaque within a lumen wall including at least one of collagen content, lipid content, calcium content, inflammation, or the relative positioning of pathophysiologic conditions within the plaque.

41.-162. (canceled)
